(19) United States
(12) Patent Application Publication
(10) Pub. No.: US 2006/0205682 A1
(43) Pub. Date: Sep. 14, 2006

(54) Antibiotic and combinations of antibiotic and symptomatic relief agent formulations

(76) Inventors: Richard Howard Roberts, Lakewood, NJ (US); Kristin Arnold, Morrisville, PA (US); Thomas J. Bryant, Winder, GA (US); Matthew William Davis, Erwinna, PA (US); Donald L. Evans, Sherman, CT (US); Gregory Keith Hayer, Pottstown, PA (US); Kurt R. Nielsen, Chadds Ford, PA (US); Jeffrey H. Ping, Braselton, GA (US); William J. Sirico, Lambertville, NJ (US)

Correspondence Address: CANTOR COLBURN, LLP, 55 GRIFFIN ROAD SOUTH, BLOOMFIELD, CT, 06002, US

(21) Appl. No.: 11/362,547

(22) Filed: Feb. 24, 2006

Related U.S. Application Data
(60) Provisional application No. 60/671,979, filed on Apr. 15, 2005.
(60) Provisional application No. 60/656,496, filed on Feb. 25, 2005.

(51) Int. Cl. A61K 31/7048; A61K 31/573; A61K 31/495; A61K 31/473; A61K 31/545; A61K 31/525; A61K 31/137; A61K 31/407; A61K 31/655

(52) U.S. Cl. 514/28; 514/29; 514/171; 514/156; 514/251; 514/290; 514/649; 514/192; 514/200; 514/210.09; 514/255.04

(57) ABSTRACT

Disclosed herein are antibiotic formulations and combinations of antibiotic and symptomatic relief agent formulations. The combinations are suitable to treat a variety of diseases, including an infection, while treating the symptoms associated with the disease. Also disclosed are methods of treating a disease or an infection and its symptoms, as well as pharmaceutical kits containing such formulations.

### CROSS-REFERENCE TO RELATED APPLICATIONS

This application claims the benefit of U.S. Provisional Application Ser. No. 60/671,979, filed Apr. 15, 2005 and U.S. Provisional Application Ser. No. 60/656,496, filed Feb. 25, 2005, both of which are incorporated by reference in their entirety.

### BACKGROUND

Antibiotics are well known active agents used to treat infections and related diseases. Depending upon the antibiotic class, any one of a variety of mechanism of action is used to target and treat the infection. For example, certain antibiotics interfere with protein synthesis, with DNA synthesis, cell wall synthesis, cell membrane permeability, and the like.

As the antibiotics target the infection or underlying disease, a patient must, however, endure any number of symptoms associated with the infection. These symptoms are often not immediately ameliorated by the antibiotic treatment itself. For example, otitis media, commonly known as an ear infection, is often accompanied by nasal congestion, congestion of the Eustachian tube, pain, or fever. Treatment of the infection as well as immediate relief of the congestion, pain, and fever would provide a comprehensive approach to the infection. Accordingly, there remains a need for dosage formulations and dosing regimens that will relieve symptoms associated with an infection or disease while an antibiotic treatment is initiated or ongoing.

As a dosing regimen becomes increasingly detailed, operator error is possible leading to the risk of over or under dosing of the antibiotic. Furthermore, confusing or complicated dosing regimens contribute to the lack of patient compliance and reduced success of the regimen. In addition to treatment of an infection or disease and the symptoms associated with the particular disease, there remains a need for convenient and easy dosing regimens of antibiotics and active agents used for symptomatic treatment.

### SUMMARY OF THE INVENTION

In one embodiment, a dosage form comprises a therapeutically effective amount of an antibiotic composition; and a therapeutically effective amount of a symptomatic relief agent; wherein the antibiotic composition is present in a first dosage form and the symptomatic relief agent is present in a second dosage form, or the antibiotic composition and symptomatic relief agent are present in a single dosage form containing both the antibiotic composition and symptomatic relief agent; with the proviso that the symptomatic relief agent is not a Cox-2 inhibitor, ephedrine, or norephedrine.

In one embodiment, a pre-packaged pharmaceutical kit comprises a plurality of dosage forms comprising an antibiotic composition; and a symptomatic relief agent; wherein the antibiotic composition is present in a first dosage form and the symptomatic relief agent is present in a second dosage form, or the antibiotic composition and symptomatic relief agent are present in a single dosage form containing both the antibiotic composition and symptomatic relief agent; wherein the plurality of dosage forms is packaged in a container with instructions for a patient to carry out a dosing regimen, and wherein the container incorporates indicia for distinguishing between the first and the second dosage forms.

In another embodiment, a method of treating a bacterial infection and an accompanying symptom comprises providing a dosing regimen to a patient comprising an antibiotic composition, and a symptomatic relief agent; wherein the antibiotic composition is present in a first dosage form and the symptomatic relief agent is present in a second dosage form, or the antibiotic composition and symptomatic relief agent are present in a single dosage form containing both the antibiotic composition and symptomatic relief agent; optionally providing indicia for distinguishing between the first and second dosage forms; providing administration instructions for the use of the first and second dosage forms or the single dosage form; wherein a plurality of the first and second dosage forms or a plurality of the single dosage form are prefilled in a container incorporating the administration instructions or the indicia.

In another embodiment, a method of treating or preventing an infection comprises providing a dosing regimen to a patient comprising an antibiotic composition, and a symptomatic relief agent; wherein the antibiotic composition is present in a first dosage form and the symptomatic relief agent is present in a second dosage form, or the antibiotic composition and symptomatic relief agent are present in a single dosage form containing both the antibiotic composition and symptomatic relief agent; optionally providing indicia for distinguishing between the first and second dosage forms; providing administration instructions for the use of the first and second dosage forms or the single dosage form; wherein a plurality of the first and second dosage forms or a plurality of the single dosage form are prefilled in a container incorporating the administration instructions or the indicia; wherein the symptomatic relief agent is a decongestant, an antihistamine, an analgesic, an antipyretic, an antitussive, an expectorant, an antidiarrhoeal, an antipruritic, a steroid, an antispasmodic, an anticholinergic, or an anesthetic.

In yet another embodiment, a method of treating otitis media comprises providing a dosing regimen to a patient comprising a morning dosage form and an evening dosage form; i) wherein the morning dosage form comprises a therapeutically effective amount of a first antibiotic composition; a therapeutically effective amount of a first symptomatic relief agent, wherein the first symptomatic relief agent is a decongestant; a pharmaceutically acceptable excipient; and optionally further comprising an antihistamine, an analgesic, a steroid, or a combination comprising at least one of the foregoing; and ii) wherein the evening dosage form comprises a therapeutically effective amount of a second symptomatic relief agent, wherein the second symptomatic relief agent is an antihistamine; a pharmaceutically acceptable excipient; optionally a therapeutically effective amount of a second antibiotic composition; and optionally further comprising an analgesic; providing indicia for distinguishing between the morning and the evening dosage forms; providing administration instructions for the use of the morning and the evening dosage forms; wherein a plurality of the morning and the evening dosage forms are prefilled in a container incorporating the administration instructions or the indicia.

In still yet another embodiment, a method of treating a urinary tract infection comprises providing a dosing regimen to a patient comprising an antibiotic composition, and a symptomatic relief agent; wherein the symptomatic relief agent is an antispasmodic, an anticholinergic, an anesthetic, an analgesic, an antipyretic, or a combination comprising at least one of the foregoing symptomatic relief agents; and wherein the antibiotic composition is present in a first dosage form and the symptomatic relief agent is present in a second dosage form, or the antibiotic composition and symptomatic relief agent are present in a single dosage form containing both the antibiotic composition and symptomatic relief agent; optionally providing indicia for distinguishing between the first and second dosage forms; providing administration instructions for the use of the first and second dosage forms or the single dosage form; wherein a plurality of the first and second dosage forms or a plurality of the single dosage form are prefilled in a container incorporating the administration instructions or the indicia.

In another embodiment, a method of treating respiratory infection comprises providing a dosing regimen to a patient comprising a morning dosage form and an evening dosage form; i) wherein the morning dosage form comprises a therapeutically effective amount of a first antibiotic composition; a therapeutically effective amount of a first symptomatic relief agent, wherein the first symptomatic relief agent is a decongestant; a pharmaceutically acceptable excipient; and optionally further comprising an antihistamine, an analgesic, a steroid, an antipyretic, an antitussive, an expectorant, or a combination thereof; and ii) wherein the evening dosage form comprises a therapeutically effective amount of a second antibiotic composition; a therapeutically effective amount of a second symptomatic relief agent, wherein the second symptomatic relief agent is an antihistamine; a pharmaceutically acceptable excipient; and optionally further comprising an analgesic, a steroid, or a combination thereof; providing indicia for distinguishing between the morning and the evening dosage forms; providing administration instructions for the use of the morning and the evening dosage forms; wherein a plurality of the morning and the evening dosage forms are prefilled in a container incorporating the administration instructions or the indicia.

In another embodiment, a dosage form comprises a therapeutically effective amount of trimethoprim; and a therapeutically effective amount of sulfamethoxazole; wherein trimethoprim is present in a first dosage form and sulfamethoxazole is present in a second dosage form, or trimethoprim and sulfamethoxazole are present in a single dosage form containing both trimethoprim and sulfamethoxazole; and wherein the first and second dosage forms or the single dosage form is provided with instructions for a patient to carry out a dosing regimen for morning and evening dosing.

In another embodiment, a dosage form comprises a therapeutically effective amount of an antibiotic composition; a therapeutically effective amount of a symptomatic relief agent; and an excipient, wherein the dosage form exhibits a dissolution profile such that at 60 minutes after combining the dosage form with 900 ml of 0.1 N HCl at 37° C.±0.5° C. according to USP 28 <711> test method 2 (paddle), 75 rpm paddle speed, at least about 70% of the antibiotic composition and symptomatic relief agent are released.

In one embodiment, a stable oral antibiotic composition comprises trimethoprim; sulfamethoxazole; a solvent; and optionally a co-solvent, wherein the oral antibiotic composition is a solution wherein the trimethoprim and sulfamethoxazole are dissolved in the solvent or co-solvent.

### BRIEF DESCRIPTION OF THE DRAWINGS

FIG. 1. An exemplary blister pack card (10) for a pharmaceutical kit containing compartments (20) labeled according to a dosing regimen (40) and perforations (30) for patient convenience;

FIG. 2. An exemplary blister pack card (10) for a pharmaceutical kit containing separate, discrete compartments for both an antibiotic composition unit form (50) and a symptomatic relief agent unit dosage form (60), where the compartments are labeled according to a dosing regimen (40).

### DETAILED DESCRIPTION OF THE INVENTION

Combinations of antibiotics and symptomatic relief agents are disclosed herein for the treatment of diseases and infections as well as the symptoms associated with the underlying disease or infection. Since the patient will experience rapid relief of one or more symptoms of the disease or infection, increased patient compliance can be realized. With better patient compliance, there is an increased likelihood that the infection can be treated more completely.

Furthermore, the combinations of the antibiotic and the symptomatic relief agent allows for an increase in the efficiency of the treatment over the efficiency of each component provided alone. Such an increase in efficiency can result without increasing possible adverse effects of the treatment.

Also disclosed herein are pharmaceutical kits to be used by patients for self administration. The kits aid in the effective implementation of antibiotics or a combination with a symptomatic relief agent according to a particular dosing regimen. The pharmaceutical kits can include instructions as a separate sheet or optionally printed on the containers housing the antibiotic alone or the combination of antibiotic and symptomatic relief agent. Such kits also improve patient compliance as all the medication for the underlying infection and symptom relief would be conveniently available in one package. With clear instructions and organization of the kit to delineate which unit dosage forms should be taken at a particular time, the patient would find even complex dosing regimens easy to follow. Such a kit will also employ fixed and variable dosing combinations in the same kit, allowing the patient during the course of the regimen to decide when to discontinue self-administration of the symptomatic relief agent. Finally, such kits would reduce patient dosing error, as there would be less chance of losing the pharmaceutical dosage forms if all dosage units are together in one package as compared to multiple packages.

Such kits also improve physician and pharmacist efficiencies. As the antibiotic composition and symptomatic relief agent is formulated or packaged together, the physician would only have to write a single prescription. Furthermore, the kit can contain dosing instructions that the physician would not need to write out for the patient. The pharmacist would only have to dispense a single kit rather than multiple, separate prescriptions.

Also provided herein are methods of treatment to treat a disease or infection and the corresponding symptoms by administering the combination of an antibiotic and a symptomatic relief agent to a patient.

Provided herein are once-daily, twice-daily, or more oral dosage forms containing a combination of a therapeutically effective amount of an antibiotic composition, and a therapeutically effective amount of a symptomatic relief agent. The dosage forms can be provided as single unit dosage forms comprising both the antibiotic composition and the symptomatic relief agent in the same dosage form. Optionally, the antibiotic and the symptomatic relief agent can each be individual, discrete dosage forms that are packaged together. Such packaging can indicate that the two or more dosage forms are to be administered together. Individual, discrete dosage forms of the antibiotic and the symptomatic relief agent may be necessitated by the compatibility of the individual components, stability of the individual components, size of the unit dosage form, production of the dosage form, and the like. Still further, the antibiotic composition and symptomatic relief agent being separate dosage forms would allow for the patient to decide when he or she will stop taking the symptomatic relief agent while continuing with the antibiotic regimen.

In another embodiment, for the first several days (e.g. first four days of treatment) the antibiotic composition and symptomatic relief agent can be in a single dosage form, forcing the patient to take both the antibiotic composition and the symptomatic relief agent at the same time. The remaining days of antibiotic and symptomatic relief agent can then be provided in separate dosage forms allowing the patient to take the symptomatic relief agent only if he or she feels the need. This scheme allows for the consumption of less medication thereby reducing the chances of side effects associated with an over consumption of medication. The various combinations allow for a simple regimen that has a high degree of patient compliance, leading to substantially improved efficacy.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. As used herein, the terms wt %, weight percent, percent by weight, etc. are equivalent and interchangeable.

The term “or” means “and/or”.

The terms “first,” “second,” and the like, “primary,” “secondary,” and the like, as used herein do not denote any order, quantity, or importance, but rather are used to distinguish one element from another.

The endpoints of all ranges directed to the same component or property are inclusive and independently combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain an asymmetric element such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Symptomatic relief agent” means an active agent, excluding antibiotics, used to treat the symptoms experienced by a patient suffering from a particular disease or infection. As used herein, “symptomatic relief agent” includes folic acid agents.

“Pharmaceutically acceptable salts” include derivatives of the disclosed compounds, wherein the parent compound is modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues such as carboxylic acids; and the like, and combinations comprising at least one of the foregoing salts. The pharmaceutically acceptable salts include non-toxic salts and the quaternary ammonium salts of the parent compound formed, for example, from non-toxic inorganic or organic acids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising at least one of the foregoing salts. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising at least one of the foregoing salts.

Exemplary pharmaceutically acceptable salts of pseudoephedrine include hydrochloride, sulfate, and equivalent non-toxic salts.

A “dosage form” means a unit of administration of an active agent. Examples of dosage forms include tablets, capsules, injections, suspensions, liquids, emulsions, creams, ointments, suppositories, inhalable forms, transdermal forms, and the like.

An “oral dosage form” means a unit of administration of an active agent prescribed or intended for oral administration. An oral dosage form may or may not comprise a plurality of subunits such as, for example, microcapsules or microtablets, packaged for administration in a single dose. The oral dosage form can be in solid or liquid form.

“Efficacy” means the ability of an active agent administered to a patient to produce a therapeutic effect in the patient.

An “effective amount” or a “therapeutically effective amount” of an active agent means a sufficient amount of the active agent to provide the desired effect, specifically a nontoxic amount. In the combination therapy, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

“Treating” and “treatment” means providing an active agent or an active agent and symptomatic relief agent combination to a patient to reduce the severity or frequency of symptoms, elimination of symptoms or underlying cause, prevention of the occurrence of symptoms or the underlying cause of such symptoms, and improvement or remediation of damage.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

The antibiotic composition, as used herein, can comprise oral antibiotics of the following general classes for example, sulfa drugs; folic acid analogs; beta-lactams, including penicillins and cephalosporins; tetracyclines; macrolides; lincosamides; streptogramins; quinolones, including fluoroquinolones; polypeptides such as polymixins; aminocyclitols; glycopeptides; oxazolidinones; and the like. “Antibiotic” means antibiotics, antibacterials, antimicrobials, antiinfectives, and the like.

Exemplary antibiotics include amoxicillin, the combination of amoxicillin and potassium clavulanate, ampicillin, the combination of ampicillin and sulbactam, atovaquone, azithromycin, carbenicillin, cefaclor, cefdinir, cefonicid, ceftibuten, cefotetan, cefpodoxime, ceftriaxone, cefuroxime, cephalexin, cephalothin, cephamycin, chlortetracycline, ciprofloxacin, clindamycin, clarithromycin, cycloserine, dalfopristin, dicloxacillin, doxycycline, erythomycin, levofloxacin, linezolid, moxifloxacin, mupirocin, oxytetracycline, penicillin, rifampin, quinupristin, the combination of dalfopristin and quinupristin, spectinomycin, sulfadiazine, sulfamethoxazole, sulfametrole, sulfamoxole, sulfalene, sulfanilamide, tetracycline, trimethoprim, the combination of trimethoprim and sulfamethoxazole, vancomycin, a combination comprising at least one of the foregoing, and the like.

Trimethoprim and sulfamethoxazole are known to inhibit bacterial growth by inhibiting the synthesis of dihydrofolic acid. Sulfonamides, such as sulfamethoxazole, are similar in structure to para-aminobenzoic acid, a substrate for the first enzyme in the tetrahydrofolic acid synthesis pathway. Trimethroprim, on the other hand, is a folate analogue and reversibly inhibits bacterial dihydrofolate reductase, a different step in the pathway. The combination of trimethoprim and sulfamethoxazole results in a synergistic affect.

In one embodiment, the formulation comprises two or more antibiotics. For example, trimethoprim or sulfarnethoxazole can be combined with an additional antibiotic. The additional antibiotic can optionally block the tetrahydrofolic acid synthesis pathway by the same or different mechanism than trimethoprim or sulfamethoxazole. Furthermore, the additional antibiotic can target those pathogens not targeted by trimethoprim or sulfamethoxazole. Such a combination of antibiotics will improve the bacterial spectrum coverage and provide a comprehensive approach to treating or preventing an infection.

The antibiotic composition can be used to treat or prevent a variety of diseases or infections including, for example, respiratory system diseases, otitis media, urinary tract infection, and the like. Antibiotics may also be prescribed as a prophylaxis after transplant surgery when a patient receives immunosuppressive therapy. Besides treatment of opportunistic infections in an immunosuppressed patient (e.g. AIDS patients, organ transplant patients, etc.), the antibiotic composition may be used for the prophylaxis of recurrent urinary tract infections, Pneumocystis carinii pneumonia, for the suppression of Burkholderia cepacia in cystic fibrosis patients, and the like.

The particular amount of antibiotic in a dosage form can be a therapeutically effective amount. Exemplary amounts of the combination of trimethoprim and sulfamethoxazole for solid oral dosage forms include, for example, about 20 to about 200 mg of trimethoprim and about 20 to about 1000 mg of sulfamethoxazole as a combination, twice a day; specifically about 40 to about 180 mg of trimethoprim and about 40 to about 900 mg of sulfamethoxazole twice a day; and more specifically about 60 to about 160 mg of trimethoprim and about 60 to about 800 mg of sulfamethoxazole once or twice a day.

In one embodiment, the antibiotic is a combination of trimethoprim and sulfamethoxazole in a weight ratio of about 1:1 to about 1:6 trimethoprim:sulfamethoxazole, specifically a weight ratio of about 1:2 to about 1:5, and more specifically about 1:3 to about 1:4. This combination can be in immediate-release or controlled-release form. A reduction in the amount of sulfamethoxazole allows for the reduction in the overall size of the form dosage, thereby allowing for convenient administration of the dosage form to the patient.

In one embodiment, a separate unit dosage form of extended-release trimethoprim can be packaged with a separate unit dosage form of sulfamethoxazole to allow, for example, for smaller, easier to swallow tablets or capsules. In a further embodiment, the symptomatic relief agent can be combined with the trimethoprim into a single dosage form, specifically a tablet or capsule. Exemplary amounts of trimethoprim in the extended-release form include, for example, about 80 to about 400 mg, specifically about 120 to about 360 mg, and more specifically about 160 to about 320 mg per dosage form.

Also provided are liquid suspensions of an antibiotic and optionally a symptomatic relief agent. In one embodiment, a suspension contains about 20 to about 80 mg of trimethoprim and about 20 to about 600 mg of sulfamethoxazole per 5 milliliters (ml) of the suspension; specifically about 40 to about 60 mg of trimethoprim and about 40 to about 400 mg of sulfamethoxazole per 5 ml of the suspension.

Also provided are liquid solutions of an antibiotic and optionally a symptomatic relief agent. Solutions differ from suspensions in that a solution is a liquid preparation that contains a solute and solvent to provide a homogenous mixture; the active agent or symptomatic relief agent can be the solute. A suspension is also a liquid preparation, but which contains particles of a substance dispersed within, but undissolved in a fluid; the active agent or symptomatic relief agent can be the undissolved substance. Generally, the liquid formulations can contain the active agents in dissolved form, undissolved form, or any combination thereof.

When the combination of trimethoprim and sulfamethoxazole is used as the antibiotic composition, oral dosage formulations can include any one of the following:

i) trimethoprim, sulfamethoxazole, and symptomatic relief agent formulated into a single dosage form (e.g., single tablet, optionally scored; capsule; liquid; etc.);

ii) trimethoprim and sulfamethoxazole formulated into a single dosage form and the symptomatic relief agent in a separate dosage form;

iii) trimethoprim and the symptomatic relief agent formulated into a single dosage form and sulfamethoxazole formulated in a separate dosage form;

iv) trimethoprim, sulfamethoxazole, and the symptomatic relief agent each formulated into a separate dosage form; and

v) trimethoprim as a separate sustained-release dosage form packaged together with sulfamethoxazole dosage form to allow for smaller overall dosage forms with or without separate symptomatic relief agent.

Otitis media, commonly known as middle ear infection, is caused by a bacteria or virus. The infection can be treated with antibiotics, although there can remain several symptoms accompanying the infection. Among the oral antibiotics available to treat otitis media, the following are generally used although others can be employed: amoxicillin, ampicillin, azithromycin, cefaclor, cefdinir, ceftibuten, ceftriaxone, erythomycin, clarithromycin, or the combination of trimethoprim and sulfamethoxazole.

Otitis media is frequently caused by blockage of the eustachian tube, which connects the nasal passages and the middle ear. Typical symptoms of otitis media include pain, fever, nasal congestion, ear pressure, or retention of fluid behind the eardrum. A symptomatic relief agent that can treat these symptoms can be used in combination with the antibiotic composition.

The antibiotic composition can be used in combination with decongestants, antihistamines, antipyretics, or analgesics to treat the symptoms of otitis media. Suitable analgesics include aspirin, diclofenac, diflunisal, etodolac, ibuprofen, indomethacin, ketoprofen, ketorolac, nabumetone, naproxen, oxaprozin, piroxicam, rofecoxib, salsalate, sulindac, tohmetin; Cox-2 inhibitors such as celecoxib, valdecoxib, other non-steroidal anti-inflammatories, acetaminophen, pharmaceutically acceptable salts, combinations thereof, and the like.

Fever can also be treated with antipyretics, many of the referenced analgesics such as aspirin, acetaminophen, and ibuprophen also function as antipyretics.

In one embodiment, the symptomatic relief agent does not comprise Cox-2 inhibitors.

Nasal congestion can be treated with decongestants or antihistamines, which can encourage drainage of fluid from the ear that resulted from the swelling of the tissues around the eustachian tube.

Antihistamines are well known active agents that block the action of histamine in the body to reduce symptoms such as congestion and runny nose. Unfortunately, many antihistamines cause drowsiness, especially when first dosed to a patient. The antihistamine can be dosed at night and the decongestant in the daytime to avoid any possible drowsiness effect during waking hours. The antihistamine dosed in the evening to pediatric patients does have the added benefit of helping the patient to fall asleep more easily. Optionally, the antihistamine and decongestant can be taken together, increasing the dosage of antihistamine at night while decreasing the decongestant dose. For the daytime, the antihistamine amount can be reduced and the dosage amount of decongestant can be increased.

Suitable antihistamines include brompheniramine, cetirizine, cetirizine hydrochloride, chlorpheniramine, chlorpheniramine maleate, clemastine, clemastine fumarate, desloratadine, diphenhydramine, fexofenadine, fexofenadine hydrochloride, loratidine, terphenadine, their pharmaceutically acceptable salts, and the like.

Decongestants are used to constrict the nasal sinus blood vessels allowing for opening of the nasal passages and relief of congestion, allowing the ear to drain fluids and relieve pressure. Suitable decongestants include sympathomimetic compounds such as phenylephrine and its pharmaceutically acceptable salts (e.g., hydrochloride, bitartrate, etc.), (+)-pseudoephedrine and its pharmaceutically acceptable salts (e.g., hydrocloride, sulfate, etc.), ephedrine and its pharmaceutically acceptable salts, and the like.

In one embodiment, the symptomatic relief agent does not comprise phenylpropanolamine, ephedrine, or norephedrine.

Therapeutically effective amounts of (+)-pseudoephedrine hydrochloride include, for example, about 30 mg or about 60 mg every four hours; or about 120 mg twice daily (up to about 240-360 mg a day). To maintain a sufficient amount of decongestant between dosing, the (+)-pseudoephedrine hydrochloride can be prepared into an extended release formulation, for example in an amount of about 120 mg.

Respiratory system diseases and the associated symptoms can be treated with the antibiotic and symptomatic relief agent combinations disclosed herein. Such diseases include Streptococcal infections, Legionnaire's disease, community-acquired pneumonia, Pneumocystis carinii pneumonia, and Haemophilus influenzae, which can manifest as upper respiratory tract infections, bronchitis, sinusitis, and the like. The common cold may lead to ear infections (otitis media) or pneumonia. Typical symptoms of the common cold include fever, coughing, congestion, sneezing, runny nose, sore throat, headache, or body aches. The antibiotic composition can be used in combination with a decongestant, an antihistamine, an antipyretic, an antitussive, or an expectorant for the treatment of a respiratory system disease.

Guaifenesin is a known expectorant which aids in relieving chest congestion by thinning or loosening the mucus in the lungs. Guaifenesin can be prepared as immediate or extended release forms as a discrete dosage forms separate from the antibiotic composition or combined in a single dosage form with the antibiotic composition. Suitable amounts of guaifensesin in each dosage form can be about 100 mg to about 1200 mg.

The antibiotic composition can be used in combination with decongestants or antihistamines to treat the symptoms of sinusitis or sinus infections. Congestion of the nose and sinuses are due to dilated blood vessels in the membranes found in the nasal area. Decongestants can be used to constrict these blood vessels allowing for opening of the nasal passages and relief of nasal congestion. Those decongestants and antihistamines previously described are suitable for the antibiotic/symptomatic relief agent combinations to treat sinusitis or other respiratory system diseases.

The antibiotic composition can further be used in combination with steroids for the treatment of sinusitis, bronchitis, or other respiratory diseases. Steroids can be used to reduce the swelling or inflammation in the nasal passages and sinuses, thereby promoting drainage from the sinuses and reducing nasal congestion. Steroids can also reduce swelling or spasm in the airways, thereby increasing airflow in respiratory diseases such as bronchitis. An exemplary oral steroid includes prednisone. Exemplary steroids suitable for nasal sprays include: beclomethasone, budesonide, flunisolide, fluticasone, mometasone, triamcinolone, and the like. When nasal spray steroids are used as the symptomatic relief agent, the antibiotic composition can be packaged together with the nasal spray as separate dosage forms (e.g., solid oral dosage form of antibiotic provided in blister packs, packaged with a steroid nasal spray in a nasal spray bottle, and including instructions for a dosing regimen). Optionally, a decongestant can be used as an additional symptomatic relief agent in combination with the antibiotic composition and steroid.

Additionally, the antibiotic composition can be used for respiratory system disease prophylaxis including Pneumocystis carinii pneumonia.

Urinary tract infections e.g., cystitis, urethritis, pyelonephritis, can be treated with antibiotics to cure the underlying cause of the infection, or for urinary tract infection prophylaxis including recurrent urinary tract infections. At the early stage of treatment, however, it would be beneficial to treat symptoms that tend to accompany the infection. Symptoms of a urinary tract infection can include the urge to urinate frequently even after the bladder is emptied, a painful burning sensation during urination, fever, cramping, or discomfort in the lower abdomen. To treat the symptoms associated with a urinary tract infection, antispasmodics, anticholinergics, anesthetics, antipyretics, or analgesics can be used in combination with the antibiotic composition.

Antispasmodics aid to relax the bladder muscle and decrease bladder contractions, which in turn helps relieve urinary frequency or urgency. Suitable antispasmodics include, for example, hyoscyamine, hyoscyamine sulfate, oxybutynin, oxybutynin hydrochloride, dicyclomine, glycopyrrolate, propantheline, atropine, scopolamine, pharmaceutically acceptable salts thereof, and the like; and a combination comprising at least one of the foregoing.

Anticholinergics can be used to inhibit bladder contractions and relieve urinary frequency or urgency. Suitable anticholinergics include, for example, flavoxate, urised, tolterodine, tolterodine tartrate, anisotropine, atropine, homatropine, clidinium, mepenzolate, methantheline, pirenzepine, propantheline, pharmaceutically acceptable salts thereof, and the like; and a combination comprising at least one of the foregoing.

Anesthetics used to relieve the symptoms of pain, burning, urgency, and frequency of urination include, for example, phenazopyridine, phenazopyridine hydrochloride, urised, tolterodine, tolterodine tartrate, pharmaceutically acceptable salts thereof, and the like; and a combination comprising at least one of the foregoing.

Also helpful in treating or preventing a urinary tract infection is the administration of acidifying agents that acidify the urine or act as anti-adhesion agents preventing bacteria from adhering or attaching to urinary tract tissues. Exemplary acidifying agents or anti-adhesion agents (hereinafter collectively referred to as “acidifying agents”) include ascorbic acid (“vitamin C”), cranberry extract, hippuronic acid, or a combination comprising at least one of the foregoing. For example cranberry extract can be formulated into about 300 to about 1200 mg capsules or tablets BID.

It is known that certain antibiotics, such as sulfa drugs or folic acid analogs, interfere with a bacteria's synthesis of tetrahydrofolic acid. Tetrahydrofolic acid is produced in the folic acid pathway from dihydrofolic acid. The synthesis of tetrahydrofolic acid is necessary for the bacteria to produce nucleic acids for its survival. As these antibiotics may interfere with the patient's own folic acid metabolism, folic acid or its analogs can be administered during or after the antibiotic treatment. Folic acid, also known as Vitamin B9 or pteroylglutamic acid, is often taken as a supplement for those individuals with folic acid deficiency. Analogs of folic acid may also be administered including 5-methyl tetrahydrofolic acid (5MeTHF), tetrahydrofolic acid (THF), and 5-formyl tetrahydrofolic acid (5CHOTHF). As used herein, “folic acid agent” is inclusive of folic acid and analogs such as those described in this paragraph.

Studies have found that women who take folic acid supplements prior to conception and during the first trimester may reduce the risk of having children with neural tube defects. Such defects include abnormalities such as spina bifida and anencephaly. The recommended amount of folic acid to prevent neural tube defects can be about 400 micrograms (0.4 milligram) to about 1000 micrograms (1.0 milligram) of folic acid daily. Such amounts can be administered in single or multiple doses.

When a folic acid agent is used as a symptomatic relief agent, it can be dosed after the treatment comprising the antibiotic composition or antibiotic and additional symptomatic relief agent (e.g., antispasmodic). For example, after four days of antibiotic composition and antispasmodic, followed by ten days of antibiotic composition alone, the folic acid agent can be dosed to the patient on the fifteenth day. This could serve to replenish folic acid in a patient who was treated with sulfamethoxazole/trimethoprim, for a urinary tract infection, which frequently occurs in women of childbearing age.

In another embodiment, the folic acid agent can be dosed during and optionally after the treatment comprising the antibiotic composition or antibiotic and additional symptomatic relief agent.

The antibiotic composition can be used in combination with an antidiarrhoeal. Antidiarrhoeals include opioid receptor agonists as well as gastrointestinal anticholinergics/antispasmodics, and the like. Exemplary antidiarrhoeals include loperamide, codeine phosphate, diphenoxylate, difenoxin, bismuth subsalicylate, Kaolin, pectin, attapulgite, dicyclomine, atropine, belladonna, belladonna extract, hyoscyamine, scopolamine, hyoscine, glycopyrrolate, mepenzolate, pharmaceutically acceptable salts thereof, or a combination comprising at least one of the foregoing antidiarrhoeals. The combination of antibiotic composition and antidiarrhoeal can be used to treat Traveler's diarrhea.

The antibiotic composition can be used in combination with an antipruritic. Suitable exemplary antipruritic agents, including anticholinergics agents, are diphenhydramine, pharmaceutically acceptable salts thereof, or a combination comprising at least one of the foregoing antipruritics.

Antibiotics are often prescribed to a patient after organ transplant surgery or to immunosuppressed patients to prevent opportunistic infections. In immunosuppressive therapy, an immunosuppressant is prescribed to prevent organ rejection. Combinations of an antibiotic and an immunosuppressant would provide both a therapeutic benefit as well as increased convenience for the patient. Exemplary immunosuppressants include azathioprine, cyclosporines, mycophenolate mofetil, prednisone, sirolimus, tacrolimus, or a combination comprising at least one of the foregoing.

The antibiotic and symptomatic relief agent can be combined into a single discrete dosage form or provided as separate, individual dosage forms. When the antibiotic and symptomatic relief agent are provided as separate dosage forms, a plurality of the forms can be packaged together to provide ease of compliance with a particular dosing regimen. Such convenient packaging is described in further detail herein.

Exemplary treatment regimens for treating infections and the symptoms associated with the underlying infection include:

1) For treatment of sinus infections: about 2 to about 20 days of antibiotic treatment and about 3 to about 14 days of decongestant or antihistamine, wherein the administration of the decongestant or antihistamine commence on the first day of antibiotic dosing and continue until the symptomatic relief agent dosing regimen is complete (e.g., about 3 to about 14 days); and
a) the decongestant or decongestant and antihistamine can be dosed in the morning while the evening dose contains only antibiotic or antibiotic and antihistamine; or
b) the dosing regimen can further include an analgesic during the first 3 to 14 days of the regimen.

2) For an infection in a woman of child bearing age treated with an antibiotic that inhibits tetrahydrofolic acid production: about 2 to about 20 days of antibiotic treatment and about 5 days of folic acid agent, wherein the folic acid agent is administered commencing on the next day after the last antibiotic dose.

3) For treatment of a urinary tract infection: about 2 to about 20 days of antibiotic treatment and about 2 to about 14 days of an antispasmodic, anticholinergic, or anesthetic, wherein the administration of the antispasmodic, anticholinergic, anesthetic, or analgesic commences on the first day of antibiotic dosing and continues until the symptomatic relief agent dosing regimen is complete (e.g., 2-14 days).

4) For immunosuppression therapy: according to the standard therapeutic treatment for immunosuppression (e.g., cyclosporin and sulfamethoxazole/trimethoprim daily).

5) For the treatment of otitis media: about 2 to about 20 days of antibiotic treatment; about 2 to about 14 days of analgesic for pain and fever; and about 2 to about 14 days of decongestant to shrink the nasal passages.

The forgoing treatment regimens are exemplary only and the total treatment time and dosing will vary from patient to patient or product to product based on medical judgment and factors particular to the patient being treated such as, for example, age, weight, physical condition of the patient, etc. Therapeutically effective doses can be determined by one of skill in the art and will depend on the severity and course of the infection, the patient's health and response to treatment, and the judgment of the treating physician. Any generally acceptable treatment regimens for the particular infection can be used with no limit on the particular dose of antibiotic or symptomatic relief agent.

The dosage form can be formulated for oral administration and may include, for example, uncoated tablets, coated tablets, modified-release tablets, gastro-resistant tablets, orodispersible tablets, effervescent tablets, chewable tablets, soft capsules, hard capsules, modified-release capsules, gastro-resistant capsules; uncoated granules, effervescent granules, coated granules, gastro-resistant granules, modified-release granules (including sprinkle forms); liquid filled gelcaps, powders for oral administration, liquid (suspension or solution), and candy forms, including hard candies and gummy or jelly forms; and further including taste-masked forms of the foregoing dosage forms. The dosage form is specifically an oral dosage form such as a solid or liquid.

The dosage form can be a releasable form, for example, instant-release, immediate-release, controlled-release, sustained-release, extended-release, delayed-release, pulsed-release, semi-delayed-release, or a combination comprising at least one of these forms (e.g., a bi-layer tablet with an immediate-release portion and an extended-release portion, or a tablet with an extended-release core that is coated with an immediate-release form). Certain releasable forms can be characterized by their dissolution profile. Dissolution profile as used herein, means a plot of the cumulative amount of active agent released as a function of time. The dissolution profile can be measured utilizing the USP Drug Release Test <724>, which incorporates standard test USP 28 or USP 29 (Test <711>). A profile is characterized by the test conditions selected. Thus the dissolution profile can be generated at a preselected apparatus type, shaft speed, temperature, volume, and composition of the dissolution media.

A first dissolution profile can be measured at a pH level approximating that of the stomach. A second dissolution profile can be measured at a pH level approximating that of one point in the intestine or several pH levels approximating multiple points in the intestine.

A highly acidic pH may simulate the stomach and a less acidic to basic pH may simulate the intestine. “Highly acidic pH” means a pH of about 1 to about 4. “Less acidic to basic pH” means a pH of greater than about 4 to about 7.5, specifically about 6 to about 7.5. A pH of about 1.2 can be used to simulate the pH of the stomach. A pH of about 6 to about 7.5, specifically about 6.8, can be used to simulate the pH of the intestine.

Release forms may also be characterized by their pharmacokinetic parameters. Release forms may also be characterized by their pharmacokinetic parameters. “Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC_{0-T} is the area under the curve of plasma concentration versus time from time 0 to time t. The AUC_{0-∞} or AUC_{0-INF} is the calculated area under the curve of plasma concentration versus time from time 0 to time infinity.

By “instant-release” is meant a dosage form designed to ensure rapid dissolution of the active agent by modifying the crystal form of the active agent to obtain a more rapid dissolution. An exemplary instant release dosage form includes an orally disintegrating dosage form.

By “immediate-release”, it is meant a conventional or non-modified release in which greater then or equal to about 75% of the active agent is released within two hours of administration, specifically within one hour of administration.

By “controlled-release” it is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, i.e., at least over several hours.

“Sustained-release” or “extended-release” include the release of the active agent at such a rate that blood (e.g., plasma) levels are maintained within a therapeutic range, but below toxic levels or a targeted level, for at least about 8 hours, specifically at least about 12 hours after administration at steady-state. The term steady-state means that a plasma level for a given active agent has been achieved and which is maintained with subsequent doses of the active agent at a level which is at or above the minimum effective therapeutic level and is below the minimum toxic plasma level or a targeted level for a given active agent.

By “delayed-release”, it is meant that there is a time-delay before significant plasma levels of the active agent are achieved. A delayed-release formulation of the active agent can avoid an initial burst of the active agent, or can be formulated so that release of the active agent in the stomach is avoided and absorption is effected in the small intestine.

A “pulsed-release” formulation can contain a combination of immediate-release, sustained-release, or delayed-release formulations in the same dosage form. A “semi-delayed-release” formulation is a pulsed-release formulation in which a moderate dosage is provided immediately after administration and a further dosage some hours after administration.

The dosage forms containing the antibiotic or symptomatic relief agents can further contain a pharmaceutically acceptable excipient well known in the art. “Pharmaceutically acceptable excipients” means any other component added to the pharmaceutical formulation other than the active agent. Excipients may be added to facilitate manufacture, enhance stability, control release, enhance product characteristics, enhance bioavailability, enhance patient acceptability, etc. Pharmaceutical excipients include carriers, fillers, binders, disintegrants, lubricants, glidants, compression aids, colors, sweeteners, preservatives, suspending agents, dispersing agents, film formers, flavors, printing inks, etc.

Binders hold the ingredients in the dosage form together. Exemplary binders include, for example, polyvinyl pyrrolidone, hydroxypropyl cellulose, hydroxypropyl methylcellulose, methylcellulose and hydroxyethyl cellulose, sugars, and combinations comprising at least one of the foregoing binders.

Disintegrants expand when wet causing a tablet to break apart. Exemplary disintegrants include water swellable substances, for example, low-substituted hydroxypropyl cellulose, e.g. L-HPC; cross-linked polyvinyl pyrrolidone (PVP-XL), e.g. Kollidon® CL and Polyplasdone® XL; cross-linked sodium carboxymethylcellulose (sodium croscarmellose), e.g. Ac-di-sol®, Primellose®; sodium starch glycolate, e.g. Primojel®; sodium carboxymethylcellulose, e.g. Nymcel ZSB10®; sodium carboxymethyl starch, e.g. Explotab®; ion-exchange resins, e.g. Dowex® or Amberlite®; microcrystalline cellulose, e.g. Avicel®; starches and pregelatinized starch, e.g. Starch 1500®, Sepistab ST200®V; formalin-casein, e.g. Plas-Vita®, and combinations comprising at least one of the foregoing water swellable substances.

Lubricants, for example, aid in the processing of powder materials. Exemplary lubricants include calcium stearate, glycerol behenate, magnesium stearate, mineral oil, polyethylene glycol, sodium stearyl fumarate, stearic acid, talc, vegetable oil, zinc stearate, and combinations comprising at least one of the foregoing lubricants. Glidants include, for example, silicon dioxide.

Certain dosage forms described herein contain a filler, such as a water insoluble filler, water soluble filler, and combinations thereof. The filler may be a water insoluble filler, such as silicon dioxide, titanium dioxide, talc, alumina, starch, kaolin, polacrilin potassium, powdered cellulose, microcrystalline cellulose, and combinations comprising at least one of the foregoing fillers. Exemplary water-soluble fillers include water soluble sugars and sugar alcohols, specifically lactose, glucose, fructose, sucrose, mannose, dextrose, galactose, the corresponding sugar alcohols and other sugar alcohols, such as mannitol, sorbitol, xylitol, and combinations comprising at least one of the foregoing fillers.

The dosage form can contain an amount of active agent sufficient to achieve a therapeutic effect with a single administration. When the formulation is a tablet or capsule, the dosage form is usually one such tablet or capsule. The frequency of administration that will provide the most effective results in an efficient manner without overdosing will vary with the characteristics of the particular active agent, including both its pharmacological characteristics and its physical characteristics such as solubility. In most cases, the dosage form will be such that effective results will be achieved with administration no more frequently than once about every four hours or more, specifically, every eight hours or more, specifically once about every twelve hours or more, and even more specifically about once every twenty-four hours or more.

The dosage form can be prepared by various conventional granulating, mixing, commutating, and fabrication techniques readily apparent to those skilled in the art of pharmaceutical formulations. Examples of such techniques include direct compression, using appropriate punches and dies, the punches and dies are fitted to a suitable rotary tableting press; injection or compression molding using suitable molds fitted to a compression unit, granulation followed by compression; and extrusion in the form of a paste, into a mold or to an extrudate to be cut into lengths.

When tablets are made by direct compression of a tableting mixture, the addition of lubricants may be helpful and sometimes important to promote powder flow and to prevent capping of the tablet (breaking off of a portion of the tablet) when the pressure is relieved. Useful lubricants are magnesium stearate (in a concentration of from 0.25 wt % to 3 wt % by weight, specifically less than 1 wt %, in the powder mix), and hydrogenated vegetable oil (e.g., hydrogenated and refined triglycerides of stearic and palmitic acids) at about 1 wt % to 5 wt %, most specifically about 2wt %. Additional excipients may be added to enhance powder flowability and reduce adherence.

Oral dosage forms may be prepared to include an effective amount of melt-extruded subunits in the form of multiparticles within a capsule. For example, a plurality of the melt-extruded muliparticulates can be placed in a gelatin capsule in an amount sufficient to provide an effective release dose when ingested and contacted by gastric fluid.

The subunits, e.g., in the form of multiparticulates, can be compressed into an oral tablet using conventional tableting equipment using standard techniques. Techniques and compositions for making tablets (compressed and molded), capsules (hard and soft gelatin) are also described in Remington's Pharmaceutical Sciences, (Aurther Osol., editor), 1553-1593 (1980).

The composition may be in the form of micro-tablets enclosed inside a capsule, e.g. a gelatin capsule. For this, a gelatin capsule employed in the pharmaceutical formulation field can be used, such as the hard gelatin capsule known as CAPSUGEL, available from Pfizer.

Optionally, the tablets can be scored to be easily broken into segments of halves or thirds. The smaller dimensions of the resulting segments allow for easier ingestion, especially for patients that have difficulty swallowing tablets. For pediatric or geriatric patients, smaller tablets or capsules can also be used. These smaller tablets or capsules are packaged together to indicate to the patient how many to take without the risk of under- or over dosing.

Certain dosage forms described herein may be coated. Coating the solid oral dosage forms also aids in easier swallowing. The coating can be a suitable coating, such as, a functional or a non-functional coating, or multiple functional or non-functional coatings. “Functional coating” means a coating that modifies the release properties of the total formulation, for example, a sustained-release coating. “Non-functional coating” means a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

The dosage forms described herein may be coated with a functional or non-functional coating. The coating may comprise about 0 wt % to about 40 wt % of the composition. The coating material may include a polymer, specifically a film-forming polymer, for example, methyl cellulose, ethyl cellulose, hydroxypropyl cellulose, hydroxypropyl methyl cellulose, hydroxybutyl methyl cellulose, cellulose acetate, cellulose propionate, cellulose acetate propionate, cellulose acetate butyrate, cellulose acetate phthalate, carboxymethyl cellulose, cellulose triacetate, cellulose sulphate sodium salt, poly(methyl methacrylate), poly(ethyl methacrylate), poly(butyl methacrylate), poly(isobutyl methacrylate), poly(hexyl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly (ethylene)high density, (poly propylene), poly(ethylene glycol, poly(ethylene oxide), poly(ethylene terephthalate), poly(vinyl alcohol), poly(vinyl isobutyl ether), poly(viny acetate), poly(vinyl chloride), polyvinyl pyrrolidone, and combinations comprising at least one of the foregoing polymers.

To provide a taste-masking effect, the polymer can be a water-insoluble polymer. Water insoluble polymers include ethyl cellulose or dispersions of ethyl cellulose, acrylic or methacrylic ester polymers, cellulose acetates, butyrates or propionates or copolymers of acrylates or methacrylates having a low quaternary ammonium content, and the like, and combinations comprising at least one of the foregoing polymers.

The inclusion of an effective amount of a plasticizer in the coating composition can improve the physical properties of the film. For example, because ethyl cellulose has a relatively high glass transition temperature and does not form flexible films under normal coating conditions, it may be advantageous to add plasticizer to the ethyl cellulose before using the same as a coating material. Generally, the amount of plasticizer included in a coating solution is based on the concentration of the polymer, e.g., most often from about 1 wt % to about 50 wt % of the polymer. Concentrations of the plasticizer, however, can be determined by routine experimentation.

Examples of plasticizers for ethyl cellulose and other celluloses include plasticizers such as dibutyl sebacate, diethyl phthalate, triethyl citrate, tributyl citrate, triacetin, and combinations comprising at least one of the foregoing plasticizers, although it is possible that other water-insoluble plasticizers (such as acetylated monoglycerides, phthalate esters, castor oil, etc.) can be used.

Examples of plasticizers for acrylic polymers include citric acid esters such as triethyl citrate NF, tributyl citrate, dibutyl phthalate, 1,2-propylene glycol, polyethylene glycols, propylene glycol, diethyl phthalate, castor oil, triacetin, and combinations comprising at least one of the foregoing plasticizers, although it is possible that other plasticizers (such as acetylated monoglycerides, phthalate esters, castor oil, etc.) can be used.

An example of a functional coating comprises a coating agent comprising a poorly-water-permeable component (a) such as, an alkyl cellulose, for example an ethylcellulose, such as AQUACOAT (a 30% dispersion available from FMC, Philadelphia, Pa.) or SURELEASE (a 25% dispersion available from Colorcon, West Point, Pa.) and a water-soluble component (b), e.g., an agent that can form channels through the poorly-water-permeable component upon the hydration or dissolution of the soluble component. Specifically, the water-soluble component is a low molecular weight, polymeric material, e.g., a hydroxyalkylcellulose, hydroxyalkyl(alkylcellulose), and carboxymethylcellulose, or salts thereof. Particular examples of these water-soluble polymeric materials include hydroxyethylcellulose, hydroxypropylcellulose, hydroxyethylmethylcellulose, hydroxypropylmethylcellulose, carboxymethylcellulose, sodium carboxymethylcellulose, and combinations comprising at least one of the foregoing materials. The water-soluble component can comprise hydroxypropylmethylcellulose, such as METHOCEL (Dow). The water-soluble component is specifically of relatively low molecular weight, specifically less than or equal to about 25,000 molecular weight, or specifically less than or equal to about 21,000 molecular weight.

In the functional coating, the total of the water soluble component (b) and poorly-water permeable component (a) are present in weight ratios (b):(a) of about 1:4 to about 2:1, specifically about 1:2 to about 1:1, and more specifically in a ratio of about 2:3. While the ratios disclosed herein are preferred for duplicating target release rates of presently marketed dosage forms, other ratios can be used to modify the speed with which the coating permits release of the active agent. The functional coating may comprise about 1 wt % to about 40 wt %, specifically about 3 wt % to about 30 wt %, more specifically about 5 wt % to about 25 wt %, and yet more specifically about 6 wt % to about 10 wt % of the total formulation.

Suitable methods can be used to apply a coating to the dosage forms. Processes such as simple or complex coacervation, interfacial polymerization, liquid drying, thermal and ionic gelation, spray drying, spray chilling, fluidized bed coating, pan coating, electrostatic deposition, and the like may be used.

The coatings may be of any thickness, specifically about 0.005 micrometers to about 25 micrometers thick and more specifically about 0.05 micrometers to about 5 micrometers.

The solid dosage form can optionally be in the form of a chewable tablet. A chewable tablet comprises a chewable base and optionally a sweetener. The chewable base comprises an excipient such as, for example, mannitol, sorbitol, lactose, or a combination comprising at least one of the foregoing excipients. The optional sweetener used in the chewable dosage form may be, for example, sucrose, liquid glucose, sorbitol, dextrose, isomalt, liquid maltitol, aspartame, acesulfame K, lactose, and combinations comprising at least one of the foregoing sweeteners. In certain cases, the chewable base and the sweetener may be the same component. The chewable base and optional sweetener may comprise about 50% to about 90% by weight of the total weight of the dosage form. In this way, “candy” forms can be prepared that are chewable.

The chewable dosage form may additionally contain preservatives, agents that prevent adhesion to the oral cavity and crystallization of sugars, flavoring agents, souring agents, coloring agents, and combinations comprising at least one of the foregoing agents. Glycerin, lecithin, hydrogenated palm oil or glyceryl monostearate may be used as a protecting agent of crystallization of the sugars in an amount of about 0.04 to about 2.0 weight % of the total weight of the ingredients, as well as to prevent adhesion to the oral cavity or to improve the soft property of the chewable dosage form.

Related to the chewable forms are candy forms prepared as pediatric dosage forms. Such candy forms include hard candy forms (e.g., lollipops, etc.) as well as soft chewy forms (e.g., gummi candies). The candy forms can be prepared in a variety of shapes and flavors to appeal to the pediatric patient. The gummi forms generally contain as a base sugar, gelatin, and flavors. An exemplary method of preparing candy forms including gummi forms are provided in U.S. Pat. No. 6,365,209.

Another oral dosage form is a non-chewable, fast-dissolving dosage form. These dosage forms can be made by methods known to those of ordinary skill in the art of pharmaceutical formulations. For example, Cima Labs has produced oral dosage forms including microparticles and effervescents, which rapidly disintegrate in the mouth and provide adequate taste-masking. Cima Labs has also produced a fast-dissolving dosage form containing an active agent and a matrix that includes a nondirect compression filler and a lubricant. Zydis® available from CardinalHealth is a rapidly dissolvable, freeze-dried, sugar matrix formulated for the preparation of rapidly dissolving tablets. U.S. Pat. No. 5,178,878 and U.S. Pat. No. 6,221,392 provide teachings regarding fast-dissolving dosage forms.

An exemplary fast-dissolving dosage form includes a mixture incorporating a water or saliva activated effervescent, a disintegration agent, and microparticles. The microparticles incorporate the active agent (e.g., antibiotic composition or symptomatic relief agent) optionally together with a protective material substantially encompassing the active agent. “Substantially encompassing” as used in this context means that the protective material substantially shields the active agent from contact with the environment outside of the microparticle. Thus, each microparticle may incorporate a discrete mass of active agent covered by a coating of the protective material, in which case the microparticle can be referred to as a “microcapsule”. Alternatively or additionally, each microparticle may have active agent dispersed or dissolved in a matrix of the protective material. The mixture including the microparticles and effervescent agent may be present as a tablet of a size and shape adapted for direct oral administration to a patient. The tablet is substantially completely disintegrable upon exposure to water or saliva. The effervescent disintegration agent is present in an amount effective to aid in disintegration of the tablet, and to provide a distinct sensation of effervescence when the tablet is placed in the mouth of a patient and contacts saliva or other source of moisture.

The effervescent sensation is not only pleasant to the patient but also tends to stimulate saliva production, thereby providing additional water to aid in further effervescent action. Thus, once the tablet is placed in the patient's mouth, it will disintegrate rapidly and substantially completely without any voluntary action by the patient. Even if the patient does not chew the tablet, disintegration will proceed rapidly. Upon disintegration of the tablet, the microparticles are released and can be swallowed as a slurry or suspension of the microparticles. The microparticles are thus transferred to the patient's stomach for dissolution in the digestive tract and systemic distribution of the active agent.

The term effervescent disintegration agent includes compounds that evolve gas. Effervescent agents may evolve gas by means of chemical reactions that take place upon exposure of the effervescent disintegration agent to water or to saliva in the mouth. The bubble or gas generating reaction is most often the result of the reaction of a soluble acid source and an alkali metal carbonate or carbonate source. The reaction of these two general classes of compounds produces carbon dioxide gas upon contact with water included in saliva.

Such water-activated materials should be kept in a generally anhydrous state with little or no absorbed moisture or in a stable hydrated form since exposure to water will prematurely disintegrate the tablet. The acid sources or acid may be any which are safe for human consumption and may generally include food acids, acid anhydrides and acid salts. Food acids include citric acid, tartaric acid, malic acid, fumaric acid, adipic acid, and succinic acids, and combinations comprising at least one of the foregoing acids. Because these acids are directly ingested, their overall solubility in water is less important than it would be if the effervescent tablet formulations of the present invention were intended to be dissolved in a glass of water. Acid anhydrides and acid of the above-described acids may also be used. Acid salts may include sodium, dihydrogen phosphate, disodium dihydrogen pyrophosphate, acid citrate salts and sodium acid sulfite.

Exemplary carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations comprising at least one of the foregoing carbonates.

The effervescent disintegration agent is not always based upon a reaction that forms carbon dioxide. Reactants that evolve oxygen or other gasses which are safe for human patients, including pediatric patients, may also be used. Where the effervescent agent includes two mutually reactive components, such as an acid source and a carbonate source, it is preferred that both components react substantially completely. Therefore, an equivalent ratio of components, which provides for equal equivalents, is preferred. For example, if the acid used is diprotic, then either twice the amount of a mono-reactive carbonate base, or an equal amount of a di-reactive base should be used for complete neutralization to be realized. However, the amount of either acid or carbonate source may exceed the amount of the other component. This may be useful to enhance taste or performance of a tablet containing an overage of either component. In this case, it is acceptable that the additional amount of either component may remain unreacted.

In general, the amount of effervescent disintegration agent useful for the formation of tablets is about 5% to about 50% by weight of the final composition, specifically about 15% to about 30% by weight thereof, and more specifically about 20% to about 25% by weight of the total composition.

More specifically, fast-dissolving tablets can contain an amount of effervescent disintegration agent effective to aid in the rapid and complete disintegration of the tablet when orally administered. By “rapid”, it is understood that the tablets disintegrate in the mouth of a patient in less than 10 minutes, and or in certain embodiments between about 30 seconds and about 7 minutes, specifically the tablets dissolve in the mouth in between about 30 seconds and about 5 minutes. Disintegration time in the mouth can be measured by observing the disintegration time of the tablet in water at about 37° C. The tablet is immersed in the water without forcible agitation. The disintegration time is the time from immersion for substantially complete dispersion of the tablet as determined by visual observation. As used herein, the term “complete disintegration” of the tablet does not require dissolution or disintegration of the microcapsules or other discrete inclusions.

The solid oral dosage forms can be taste-masked to increase patient compliance. Taste-masking can be effected by coating the solid dosage form as described above.

Suitable taste-masking technologies that can be used are found in U.S. Pat. No. 6,139,865 incorporated herein by reference in its entirety.

Sprinkle dosage forms include particulate or pelletized forms of active agent, optionally having functional or non-functional coatings, with which a patient or a caregiver can sprinkle the particulate/pelletized dose into drink or onto soft food. A sprinkle dosage form may comprise particles of about 10 to about 100 micrometers in their major dimension. Sprinkle dosage forms may be in the form of optionally coated granules or as microcapsules. Specifically the sprinkle dosage forms are taste masked. See U.S. Pat. No. 5,084,278, which is hereby incorporated by reference for its teachings regarding microcapsule formulations, which may be administered as sprinkle dosage forms.

Liquid dosage forms of an antibiotic or combination of antibiotic and symptomatic relief agent thereof can be in the form of solutions or as a suspension. Furthermore, the liquid dosage form can be formulated to provide adequate taste-masking. A taste-masked liquid dosage form may comprise a suspension of taste-masked particles (e.g., microparticles). The use of polymeric coatings on the active agent microparticles, which inhibit or retard the rate of dissolution and solubilization of the active agent is one means of overcoming the taste problems with delivery of active agents in suspension. The polymeric coating allows time for the particles to be swallowed before the taste threshold concentration is reached in the mouth.

A taste-masked liquid dosage form thus comprises the active agent, a polymer encapsulating the active agent, and a suspending medium for suspending the encapsulated active agent. The active agent can be taste-masked by the polymer or polymer and suspending medium.

The active agent (antibiotic or antibiotic and symptomatic relief agent) of the taste-masked form may be in the form of its neutral or salt form and may be in the form of particles, crystals, microcapsules, granules, microgranules, powders, pellets, amorphous solids or precipitates. The particles may further include other functional components. The active agent may have a defined particle size distribution, specifically in the region of about 0.1 to about 500 μm, more specifically about 1 to about 250 μm, and most specifically about 10 to about 150 μm, where there is acceptable mouth feel and little chance of chewing on the residual particles and releasing the active agent to taste.

The taste-masked liquid dosage form may include, along with the active agent, other functional components present for the purpose of modifying the physical, chemical, or taste properties of the active agent. For example the antibiotic may be in the form of ion-exchange or cyclodextrin complexes or may be included as a mixture or dispersion with various additives such as waxes, lipids, dissolution inhibitors, taste-masking or -suppressing agents, carriers or excipients, fillers, and combinations comprising at least one of the foregoing components.

The pharmaceutically active agent or the active agent particle may be suspended, dispersed or emulsified in the suspending medium after encapsulation with the polymer. The suspending medium may be a water-based medium, but may be a non-aqueous carrier as well. The taste-masked liquid dosage form may further include other optional dissolved or suspended agents to provide stability to the suspension. These include suspending agents or stabilizers such as, for example, methyl cellulose, sodium alginate, xanthan gum, (poly)vinyl alcohol, microcrystalline cellulose, colloidal silicas, bentonite clay, and combinations comprising at least one of the foregoing agents. Other agents used include preservatives such as an alkyl paraben, e.g. methyl, ethyl, propyl and butyl parabens; sweeteners such as sucrose, sucralose, saccharin sodium, aspartame, acesulfame K, mannitol; flavorings such as grape, cherry, orange, peppermint, menthol, root beer, wintergreen, and vanilla flavors; and antioxidants or other stabilizers; and a combination comprising at least one of the foregoing agents. A suitable flavorant includes the liquid flavorants available from Unicorn Natural Products Ltd. under the tradename MagnaSweet (e.g. MagnaSweet 110, 115, 118, or 136).

Encapsulation of the microparticle or active agent particle by the polymer may be performed by a method such as suspending, dissolving, or dispersing in a solution or dispersion of polymer coating material and spray drying, fluid-bed coating, simple or complex coacervation, coevaporation, co-grinding, melt dispersion and emulsion-solvent evaporation techniques, and the like.

Alternatively, the polymer coated active agent powder can be applied for the preparation of reconstitutable powders, ie; dry powder active agent products that are reconstituted as suspensions in a liquid vehicle such as water before usage. The reconstitutable powders have a long shelf life and the suspensions, once reconstituted, have adequate taste-masking.

Suitable liquid taste-masked dosage forms include those disclosed in U.S. Pat. No. 6,197,348.

In one embodiment, the antibiotic or antibiotic and symptomatic relief agent is in the form of a solution for oral administration rather than a suspension or dispersion. Such a form does not result in the settling of active agent over time, thereby allowing for the complete ingestion of the appropriate dose of active agent. The oral solution can contain the antibiotic or symptomatic relief agent, solvent, and optional additives such as a co-solvent (or “solubility enhancer”), colorant, flavorant, sweetener, preservative, or a combination comprising at least one of the forgoing additives.

The solvent can be any pharmaceutically acceptable liquid that can dissolve the active agent (antibiotic or symptomatic relief agent) to form a solution, optionally with the aid of a co-solvent. Exemplary solvents include, water, pharmaceutically acceptable hydroxylic solvents including alcohols such as ethanol, glycerin, polyethylene glycol (e.g. PEG 200, 300, 400, 600), propylene glycol, and the like, and combinations comprising at least one of the foregoing. “Co-solvent” means any agent that increases the solubility of the active agent, including for example, additional solvents used in combination with the primary solvent, solubilizers, complexation agents, and the like. Exemplary co-solvents include the solvents previously listed, as well as benzyl alcohol, cyclodextrins (e.g., alpha, beta, gamma, hydroxypropyl-beta, B-cyclodextrin sulfobutyl ether sodium), ethanol, N-methyl-2-pyrrolidone, polyoxyethylene sorbitan fatty acid esters, povidone (e.g. PVP K-29/32), 2-pyrrolidone, sodium lauryl sulfate, sorbitan fatty acid esters, and the like, and combinations comprising at least one of the foregoing.

Exemplary sweeteners, flavorants, or preservatives, are those previously disclosed herein with regard to the taste-masked dosage forms. Colorants suitable for the preparation of pharmaceutical formulations may be used including those available from Colorcon, Inc. The exact range of amounts for each type sweeteners, flavorants, preservatives, or colorants can be selected by those skilled in the art.

The oral solution can be prepared by conventional techniques, generally by dissolving the active agent in the solvent and optional co-solvent with mixing. The remaining optional components may be added to the solution sequentially or at the start of the mixing process. To aid in dissolving the components heat may be applied as long as the stability and integrity of the solution components is not compromised. Additionally, sonication can be used to promote dissolution. Any other physical process to aid dissolution can be employed as is known to those skilled in the art. Once the oral solution is formed, it can optionally be filtered to remove any undissolved component.

If necessary buffers may be added to the oral solution to obtain a targeted pH. Adjustment of pH may aid in dissolving the components of the oral solution. Exemplary pH for the solution can be about 4 to about 8.5, specifically about 5 to about 7.5, more specifically about 5.5 to about 7.0, and yet more specifically about 6.0 to about 6.5. Exemplary buffers include pharmaceutically acceptable acid buffers including citric acid/sodium citrate buffer solutions or sodium phosphate dibasic and monobasic buffers.

In one embodiment, the oral solution comprises trimethoprim and sulfamethoxazole in an aqueous system. In another embodiment, the oral solution containing trimethoprim and sulfamethoxazole is formulated to contain about 20 mg to about 60 mg of trimethoprim per 5 ml of oral solution, specifically about 30 mg to about 50 mg of trimethoprim per 5 ml of oral solution, and more specifically about 35 mg to about 45 mg of trimethoprim per 5 ml of oral solution. Such an oral solution is formulated to contain about 20 mg to about 325 mg of sulfamethoxazole per 5 ml of oral solution, specifically about 125 mg to about 275 mg of sulfamethoxazole per 5 ml of oral solution, and more specifically about 175 mg to about 225 mg of sulfamethoxazole per 5 ml of oral solution.

In another embodiment, the oral solution is formulated to comprise trimethoprim and sulfamethoxazole in a weight ratio of about 1:1 to about 1:6, specifically about 1:2 to about 1:5, and yet more specifically about 1:3 to about 1:4.

The oral solution of an antibiotic composition, or an antibiotic composition and symptomatic relief agent, provides a unique formulation that can avoid the potential inhomogeneity problems found in suspension formulations. Additionally, shelf stable and palatable solution forms can be prepared.

In one embodiment, the oral solution comprises an antibiotic composition and a symptomatic relief agent, wherein the symptomatic relief agent is a decongestant or an analgesic/antipyretic. The antibiotic composition specifically comprises trimethoprim and sulfamethoxazole.

In one embodiment, the oral solution comprises trimethoprim and sulfamethoxazole in an aqueous solvent, wherein the oral solution is substantially stable for 28 days at 60° C. as measured for degradation of active agent or the formation of impurities. Specifically, the oral solution contains less than or equal to about 2.0 wt/v % impurities, specifically less than or equal to about 1.0 wt/v %, and more specifically less than or equal to about 0.1 wt/v % of impurities based on the total weight of the oral solution.

In another embodiment, the oral solution comprises less than or equal to about 20 w/v % water based on the total solution, specifically less than or equal to about 10 w/v % water, more specifically less than or equal to about 8 w/v % water, and yet more specifically less than or equal to about 5 w/v % water.

The oral antibiotic solution or symptomatic relief agent is particularly useful for treating pediatric and geriatric patients who have trouble swallowing solid oral dosage forms. The oral solution form as opposed to the oral suspension form is also more palatable and pleasing to the patient as there are no fine particles to adhere to the patient's tongue. Palatability is important for patient compliance, especially when the dosing regimen extends for days or weeks.

In one embodiment, the oral antibiotic solution comprises trimethoprim and sulfamethoxazole for the prophylaxis of infections, specifically opportunistic infections in immunosuppressed patients, otitis media, urinary tract infections, respiratory system illness such as Pneumocystis carinii pneumonia, to suppress the growth of Burkholderia cepacia in cystic fibrosis patients, and the like.

In one embodiment, the oral antibiotic solution comprises trimethoprim and sulfamethoxazole for the prophylaxis of otitis media wherein the oral antibiotic solution is administered to an adult patient at the first sign of an upper respiratory tract infection, and wherein about 20 to about 160 mg of trimethoprim and about 20 to about 800 mg of sulfamethoxazole is administered every 12 hours for about 10 days. In another embodiment, the oral antibiotic solution comprises trimethoprim and sulfamethoxazole for the prophylaxis of otitis media wherein the oral antibiotic solution is administered to a pediatric patient at the first sign of an upper respiratory tract infection, and wherein about 4 mg/kg of body wt or greater of trimethoprim and about 4 mg/kg or greater, specifically about 20 mg/kg or greater of sulfamethoxazole is administered every 12 hours for about 10 days.

In one embodiment, the oral antibiotic solution comprises trimethoprim and sulfamethoxazole for the prophylaxis of recurrent urinary tract infections wherein the oral antibiotic solution is administered once daily for a minimum of three times a week or postcoitally. A daily dose of the oral antibiotic solution may contain about 20 mg to about 80 of trimethoprim, specifically about 40 mg; and about 20 to about 300 mg sulfamethoxazole, specifically about 200 mg.

In one embodiment, the oral antibiotic solution comprises trimethoprim and sulfamethoxazole for the prophylaxis of an opportunistic infection (e.g. Pneumocystis carinii pneumonia) in an immunosuppressed patient (e.g. organ transplant patient) wherein the oral antibiotic solution is administered once every 24 hours. Specifically, the daily dose of the oral antibiotic solution may contain about 20 mg to about 400 mg of trimethoprim, specifically about 160 to about 320 mg; and about 20 to about 1800 mg sulfamethoxazole, specifically about 800 to about 1600 mg. In pediatric patients, for Pneumocystis carinii pneumonia prophylaxis, about 20 to about 150 mg of trimethoprim and about 20 to about 750 mg of sulfamethoxazole can be administered per day in equally divided doses twice a day, on 3 consecutive days per week.

In one embodiment, the oral antibiotic solution comprises trimethoprim and sulfamethoxazole for the prophylaxis of Burkholderia cepacia in a cystic fibrosis patient wherein about 4 mg/kg of body wt or greater of trimethoprim and about 4 mg/kg of body wt or greater, specifically about 20 mg/kg or greater of sulfamethoxazole is administered every 12 hours.

In one embodiment, the oral solution is free of povidone, a block copolymer (e.g. ethylene oxide-propylene oxide block copolymer), glycerol formal (a.k.a. 1,3-dioxan-5-ol), a triglyceride, or a dioctylsulfosuccinate or an alkali metal salt thereof.

The liquid, powder, and sprinkle forms are ideal for pediatric or geriatric patients that have difficulty ingesting solid dosage forms such as tablets or capsules. The antibiotic or antibiotic/symptomatic relief agent in liquid, powder, or sprinkle form can be prepackaged in unit dosage forms such as dropper bottles, eyedroppers, syringes, ampules, vials and the like, each one containing the proper amount of active agent for a particular dosing regimen. For added convenience, the dropper bottles, eyedroppers, syringes, ampules, vials and the like can be pre-filled with single or multiple doses of the active agent(s), specifically single use. Pre-filled containers can be prepared “ready-to-use” thereby providing accurate administration of a particular dose. The multidose containers can optionally further contain graduated units (e.g., marked intervals in milliliters or milligram measurements) for easy control over the administration of the desired dose. As the pre-filled containers may store an active agent for an extended period of time, the components of the containers can be prepared from materials that will not be substantially affected by contact with the active agents and inactive ingredients.

Also included herein are pharmaceutical kits useful, for example, for the treatment of an infection and amelioration of symptoms associated with the infection, which comprise a container containing a combination comprising a therapeutically effective amount of an antibiotic and a therapeutically effective amount of a symptomatic relief agent. The kits may further comprise a conventional pharmaceutical kit component, such as, for example, a container to aid in facilitating compliance with a particular dosing regimen; a carrier; printed instructions, either as inserts or as labels, indicating quantities of the components to be administered, guidelines for administration, or guidelines for mixing or measuring the components.

The containers to aid in facilitating the dosing regimen can include bubble or blister packs where each daily dosage is arranged in a geometric orientation, for example, in linear, curvilinear, circular, rectangle, etc. or any other configuration that aids or instructs the patient to conform to a particular dosing regimen. The containers or unit dosage forms themselves can include written instructions, color codes, or any other indicia or formats to inform the patient of the particular dosing regimen.

The dosing regimen may comprise a blister pack card, wherein each card can be perforated along the daily dose for patient convenience. Suitable blister packs that can be arranged in a variety of configurations to accommodate a particular dosing regimen are well known in the art or easily ascertained by one of ordinary skill in the art.

In one embodiment, the pharmaceutical kits can comprise up to seven dosage units or multiples thereof, wherein the dosage units are daily dosage units comprising a targeted amount e.g. the full day's amount, half day's amount, a third of the day's amount, etc. of the antibiotic composition and symptomatic relief agent either as a single dosage form or as separate dosage forms.

In one embodiment, a pharmaceutical kit is provided that contains a plurality of oral dosage forms for self administration; a container, specifically sealed, for housing the dosage forms during storage and prior to use; and instructions for a patient to carry out administration. The instructions will typically be written instructions on a package insert, a label, or on other components of the kit, and the oral dosage forms are as described herein. Each dosage form may be individually housed, as in a sheet of a metal foil-plastic laminate with each dosage form isolated from the others in individual compartment (e.g., cells, bubbles, or blisters), or the dosage forms may be housed in a single container, e.g. as in a plastic bottle. The packaging can be in the form of a cardboard or paper box, a plastic or foil pouch, blister packs, cards of blister packs, etc.

In one embodiment, the pharmaceutical kit comprises a double dose of antibiotic composition as the first dose (“loading dose”).

In another embodiment, the instructions are provided on the container itself by the manufacturer or provided by the pharmacist at the time the prescription is filled. The latter option would allow for the application of the instructions (e.g. by placing a sticker on the particular container, folding a flap to expose the proper instruction, use of color codes, removable label, etc.) depending upon the time of day the dosing regimen commences, e.g. once a day, twice a day (morning or evening), three times a day, four times a day, or more. This approach would allow flexibility and convenience for using one package regardless of the time the patient commences the dosing regimen.

In one embodiment, a pharmaceutical kit comprises an antibiotic for morning and evening dosing to provide an increase in patient compliance as well as ease of dispensing the appropriate dosing regimen. Such an embodiment can comprise trimethoprim or sulfamethoxazole as the antibiotic in a suitable dosage pack such as a blister pack with clearly delineated dosing regimen informing the patient what she should take and at what time. The combination of trimethoprim and sulfamethoxazole can be as a single dosage form or each antibiotic in separate dosage forms.

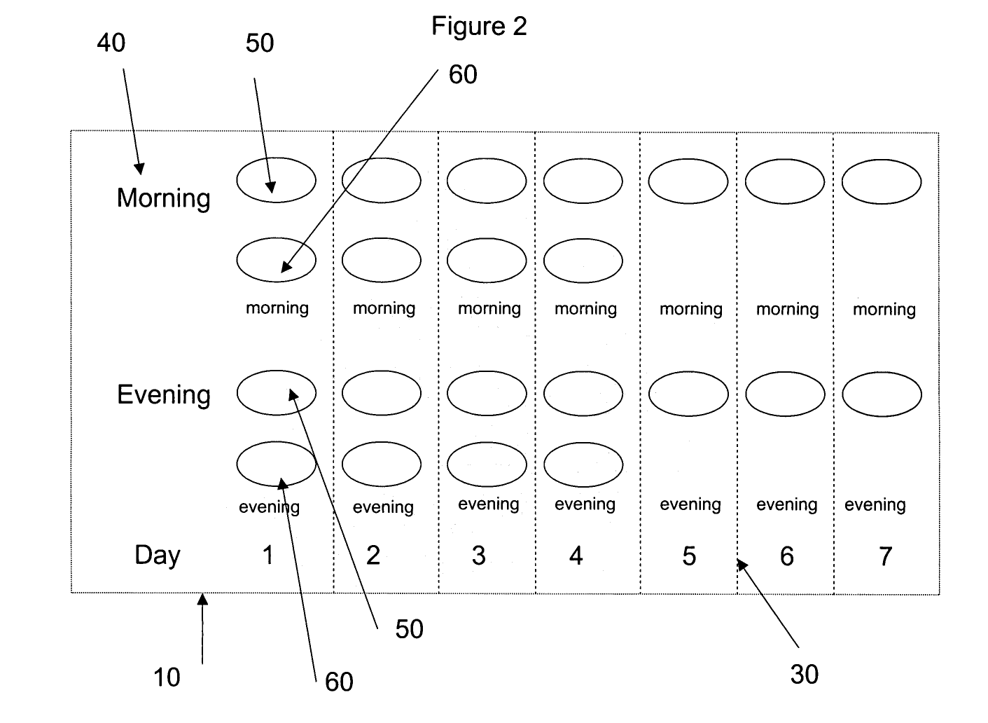

In one embodiment, a kit comprises an antibiotic and pseudoephedrine hydrochloride in separate dosage forms. Such an embodiment can comprise trimethoprim or sulfamethoxazole as the antibiotic in tablet dosage form in a suitable dosage pack such as a blister pack with clearly delineated dosing regimen informing the patient what she should take and at what time. The pseudoephedrine hydrochloride can be packaged as a separate tablet dosage form with the antibiotic, specifically in the same blister card. In an exemplary kit, solid oral dosage tablets of sulfamethoxazole and trimethoprim, in amounts of about 400 and 80 mg or 800 and 160 mg, respectively can be packaged in a blister pack card such as the one found in FIG. 2 in compartment (50) while solid oral dosage tablets of pseudoephedrine hydrochloride, (e.g. extended release form of 120 mg) can be packaged in compartment (60).

A method of treating an infection and the corresponding symptoms associated with the particular infection comprises administering a therapeutically effective dose of an antibiotic composition and a symptomatic relief agent.

A method for treating a patient with an infection involves the daily oral administration of a dosage form, specifically a single dosage form containing therapeutically effective amounts of an antibiotic composition and a symptomatic relief agent.

In one embodiment, oral tablet dosage forms comprising a therapeutically effective amount of an antibiotic composition and a therapeutically effective amount of a symptomatic relief agent exhibit a dissolution profile of at least about 70% release of antibiotic composition and symptomatic relief agent after 60 minutes in 900 ml of 0.1N HCl according to USP 28 <711> apparatus 2 (paddles), 75 rpm paddle speed and a temperature of 37° C.±0.5° C. Other dosage forms exhibit a dissolution profile of at least about 80%, more specifically at least about 90%, yet more specifically at least about 95%, and still more specifically at least about 99% release of antibiotic composition and symptomatic relief agent after 60 minutes in 900 ml of 0.1N HCl according to USP 28 <711> apparatus 2 (paddles), 75 rpm paddle speed and a temperature of 37° C.±0.5° C.

In another embodiment, a process for preparing an oral solid dosage form comprises intimately mixing an antibiotic composition and a symptomatic relief agent with a pharmaceutically acceptable excipient to form a mixture; and filling capsules with the mixture to form the dosage form; or compressing the mixture into tablets to form the dosage form.

The following examples further illustrate the invention but, of course, should not be construed as in any way limiting its scope.

### EXAMPLES

### Example 1

### Pharmaceutical Kits Containing Antibiotic and Decongestant/Antihistamine Combinations as Single Dosage Forms (Tablets)

Tablets containing an antibiotic and a decongestant or antihistamine contain the components listed in Table 1 below along with pharmaceutically acceptable excipients chosen to provide a material that can be formed by direct compression.

TABLE 1

| Component (amounts in | | | | |
|---|---|---|---|---|
| milligrams) | | | | |
| Tablet core components | A | B | C | D |
| trimethoprim | 80 | 80 | 80 | 80 |
| sulfamethoxazole | 400 | 400 | 400 | 400 |
| pseudoephedrine | — | — | 120* | 120* |
| hydrochloride | | | | |
| Diphenhydramine | — | 50 | — | 50 |
| hydrochloride | | | | |

*components formulated into an extended-release pseudoephedrine hydrochloride unit

The tablets containing the diphenhydramine hydrochloride is formulated for immediate-release of the diphenhydramine component, while the tablets containing the pseudoephedrine hydrochloride is formulated as an extended-release for the pseudoephedrine component as a unit which is later compressed with the antibiotic or antibiotic and diphenhydramine hydrochloride tablet unit to form a single tablet.

All components of each formulation are blended and compressed into tablets using a tablet press. The tablets are then film coated.

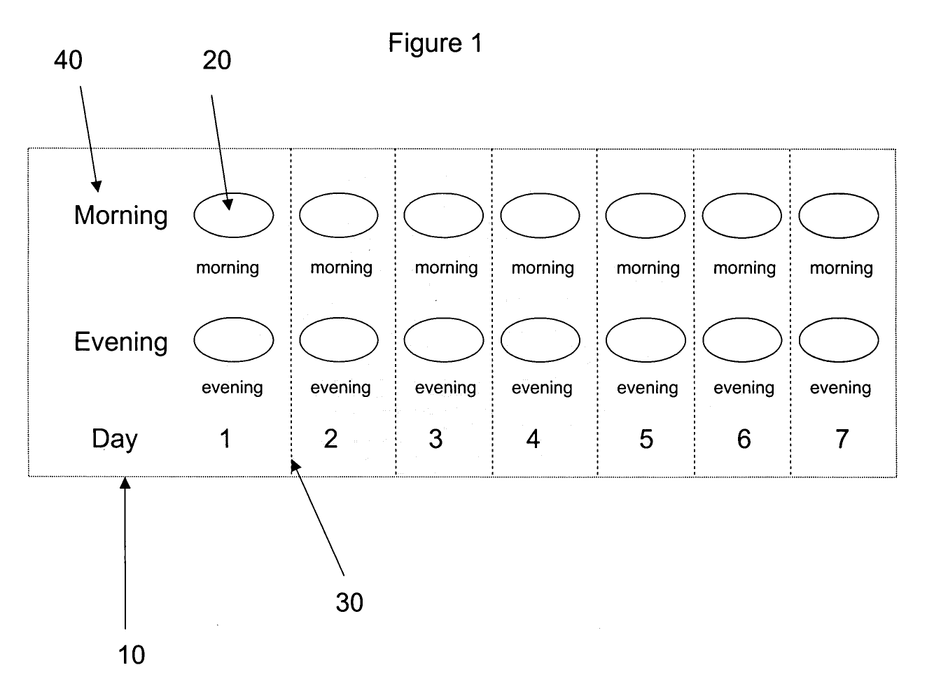

A pharmaceutical kit is prepared containing blister pack cards as exemplified in FIG. 1. The tablets prepared from formulation C, containing decongestant, are packaged in the morning dose compartments for the first 4 days while the tablets for formulation B or D, containing antihistamine or antihistamine and decongestant are packaged for the evening dose. From the fifth day forward, both the morning and evening dose compartments contain formulation A, antibiotic only.

### Example 2

### Pharmaceutical Kit Containing Antibiotic and Decongestant/Antihistamine Combinations as Separate, Discrete Dosage Forms (Tablets)

Tablets containing an antibiotic and a decongestant or antihistamine contain the components listed in Table 2 below:

TABLE 2

| Component (amounts in | | | |
|---|---|---|---|
| milligrams) | | | |
| Tablet core components | E | F | G |
| trimethoprim | 160 | — | — |
| sulfamethoxazole | 800 | — | — |
| pseudoephedrine hydrochloride | — | — | 120* |
| chlorpheniramine maleate | — | 12 | — |

*components formulated into an extended-release pseudoephedrine hydrochloride unit

All components of each formulation are blended and compressed into tablets using a tablet press. The tablets are then film coated.

A pharmaceutical kit is prepared containing blister pack cards as exemplified in FIG. 2. The tablets prepared from formulation G, containing decongestant are packaged in one of the morning dose compartments for the first 4 days while the tablets for formulation F, containing antihistamine are packaged in one of the compartments for the evening dose, also for the first 4 days. The remaining compartments contain formulation A, antibiotic only.

### Example 3

### Dosing Regimen for Urinary Tract Infection

A 28-year-old female patient suffering from a urinary tract infection is given a tablet containing 160 mg trimethoprim, 800 mg sulfamethoxazole, and 3 mg of oxybutynin hydrochloride BID orally for 4 days. Commencing on the fifth day, the patient is administered a tablet containing 160 mg trimethoprim and 800 mg sulfamethoxazole BID for 10 days. Administration of the tablets results in the treatment of the infection while reducing or eliminating the symptoms of urgency to urinate at the early stage of the treatment.

### Example 4

### Dosing Regimen for Urinary Tract Infection

A 23-year-old female patient suffering from a urinary tract infection is given a tablet containing 160 mg trimethoprim, 800 mg sulfamethoxazole, and 3 mg of oxybutynin hydrochloride BID orally for 4 days. Commencing on the fifth day, the patient is administered a tablet containing 160 mg trimethoprim and 800 mg sulfamethoxazole BID for 10 days for a total of 14 days. On the fifteenth day, the patient is administered a tablet containing 400 micrograms of folic acid QD for five days. Administration of the tablets results in the treatment of the infection while reducing or eliminating the symptoms of urgency to urinate at the early stage of the treatment. Additionally, at the late stage of the treatment, folic acid levels are restored.

### Example 5

### Dosing Regimen for Urinary Tract Infection

A 30-year-old female patient suffering from a urinary tract infection is given a tablet orally, the tablet contains 80 mg trimethoprim, 400 mg sulfamethoxazole, and 2 mg of tolterodine tartrate BID for 4 days. Commencing on the fifth day, the patient is administered a tablet containing 80 mg trimethoprim and 400 mg sulfamethoxazole for 10 days, for a total of 14 days. Administration of the tablets results in the treatment of the infection while reducing the symptom of urgency to urinate during the early stage of the treatment.

### Example 6

A 50-year-old male patient suffering from a sinus infection is given a blister pack organized for daily dosing, including an oral tablets for the morning and an oral tablets for the evening. The tablets for the morning contain 80 mg trimethoprim, 400 mg sulfamethoxazole, and 120 mg of pseudoephedrine hydrochloride for 4 days. The tablets for the evening contain 80 mg trimethoprim, 400 mg sulfamethoxazole, and 10 mg cetirizine hydrochloride for 4 days. The blister pack contains tablets for an additional 10 days for morning and evening dosing containing 80 mg trimethoprim, and 400 mg sulfamethoxazole. Administration of the tablets results in the treatment of the infection while reducing the symptoms of sinus pressure, pain, and congestion.

### Example 7

A 5-year-old male patient suffering from otitis media and having symptoms of eustachian tube congestion is given an oral tablet containing 80 mg trimethoprim, 400 mg sulfamethoxazole, and 15 mg of pseudoephedrine hydrochloride as a morning dose and tablets containing 80 mg trimethoprim, 400 mg sulfamethoxazole, and 12.5 mg diphenhydramine hydrochloride for an evening dose for 4 days, followed by the administration of oral tablets containing 80 mg trimethoprim, 400 mg sulfamethoxazole BID for 10 days. Administration of the tablets results in the treatment of the infection and drainage of the fluids in the eustachian tube and relief of pain in the ear during the early stage of the treatment.

### Example 8

Tablets containing 400 mg sulfamethoxazole, 80 mg trimethoprim, and 80 mg pseudoephedrine hydrochloride are tested for dissolution profiles. The USP 28 <711> test method 2 (paddle) is used with the following parameters:

900 ml 0.1 N HCl dissolution medium

75 rpm paddle speed

60 minutes time

37° C.±0.5° C. temperature

Samples are taken at 60 minutes after the addition of the tablets to the dissolution medium. The samples are analyzed using high performance liquid chromatography analysis against standard solutions of active agents to determine the percent release of active agents in the dissolution medium.

The tablets exhibit a dissolution profile of greater than 70% release of active agents at 60 minutes.

### Example 9

Tablets containing 800 mg sulfamethoxazole, 160 mg trimethoprim, and 50 mg of diphenhydramine hydrochloride are tested for dissolution rates according to the procedure in Example 8. The tablets exhibit a dissolution profile of greater than 97% release of active agents at 60 minutes.

### Example 10

### Antibiotic Oral Solutions, (Cyclodextrin)

Several antibiotic oral solutions are prepared containing the antibiotic compounds trimethoprim or sulfamethoxazole in a 5% potassium phosphate buffer solution containing gamma-cyclodextrin as a solubility enhancer. A 5 ml aliquot of 5% potassium phosphate buffer is added to 500 mg of gamma-cyclodextrin and sonicated until a clear solution is formed. Sulfamethoxazole is dissolved in the buffer-cyclodextrin solution and sonicated for 30 minutes until a precipitate or saturated solution is formed. The process is repeated with two additional amounts of gamma-cyclodextrin. Known aliquots of each solution are placed in a 2 cm cell and analyzed using a UV-Vis Spectrophotomer between the wavelengths 200-400 nm. The following results are obtained:

TABLE 3
Solubility mg/100 ml

| | pH 8, | pH 8, | pH 8, | pH 8, |
|---|---|---|---|---|
| | no | 500 mg | 750 mg | 1000 mg |
| | cyclodextrin | cyclodextrin | cyclodextrin | cyclodextrin |
| Sulfa- | 1030 | 1181 | 1366 | 2000 |
| methoxazole | | | | |

The preparation of trimethoprim-cyclodextrin solutions is prepared according to the procedure described above for sulfamethoxazole-cyclodextrin solutions. The following results are obtained:

TABLE 4
Solubility mg/100 ml

| | pH 8, | pH 8, | pH 8, | pH 8, |
|---|---|---|---|---|
| | no | 500 mg | 750 mg | 1000 mg |
| | cyclodextrin | cyclodextrin | cyclodextrin | cyclodextrin |
| trimethoprim | 35 | 100 | 125 | 160 |

### Example 11

### Antibiotic Oral Solutions, (Glycerin and PEG 400)

Several antibiotic oral solutions are prepared containing an antibiotic combination of trimethoprim and sulfamethoxazole in the amounts provided in Table 5.

TABLE 5

| Component (amounts in | | | | | | | |
|---|---|---|---|---|---|---|---|
| % wt/v unless otherwise | | | | | | | |
| stated) | H | I | J | K | L | M | N |
| trimethoprim (mg/5 ml) | 40 | 40 | 40 | 40 | 40 | 40 | 40 |
| sulfamethoxazole | 200 | 200 | 200 | 200 | 200 | 200 | 200 |
| (mg/5 ml) | | | | | | | |
| Polyethylene glycol 400 | 25% w/w | 25% | 25% | 25% | 25% | 25% | 25% |
| (PEG400) | | | | | | | |
| methyl paraben | — | 0.15 | 0.15 | 0.15 | 0.15 | 0.075 | 0.075 |
| propylparaben | — | 0.03 | 0.03 | 0.03 | — | 0.015 | — |
| Glycerin | 50% w/w | q.s. | q.s. | q.s. | q.s | q.s | q.s |
| Purified (DI) Water | q.s. | 10 | 10 | 10 | 10 | 10 | 10 |
| Flavorants | — | 3.27 | 3.27 | 3.27 | 3.27 | 3.27 | 3.27 |
| Colorant | — | 5.5 ppm | 0.2 | 0.1 | 0.0055 | 0.0055 | 0.0055 |
| Sweeteners (sucralose | — | 3.0 | 3.0 | 3.0 | 3.0 | 3.0 | 3.0 |
| and sodium saccharin) | | | | | | | |

The components of each formulation are combined and sonicated to aid in the dissolution of the components to form a clear solution. If necessary, aliquots of a buffer solution can be added to the formulation to achieve a targeted pH of about 6 to about 6.5. The resulting solutions are optionally filtered through a 0.45 micrometer Nylon filter to remove trace insoluble material that may be present to result in solutions having a total impurity of less than 0.1 % w/v of individual unidentified impurity. Formulations I-K are found to have a pleasing taste.

The oral solution of Formulation I was tested for stability upon acid and base stress conditions. For control, each of trimethoprim and sulfamethoxazole is prepared into samples free of the pharmaceutical excipients of Formulation I. Stress of sulfamethaxazole and trimethoprim is performed using 1N HCl and 1N NaOH. Stock solutions of each sulfamethaxazole and trimethoprim at 1000% LC dissolved in 100% MeOH are prepared. Dilutions are performed 5:50 for a total of 3 replicates. One mL of acid and base is added to the appropriate flask while the control (no added acid or base) is brought to volume using mobile phase (20% acetonitrile/80% TEA-Acetic Acid Buffer pH 5.9). The solutions are then placed into an oven at 80° C. for 3 hours, cooled, neutralized with 1.0 mL of the opposite acid/base, and brought to volume using mobile phase. The stressed solutions are analyzed by High Performance Liquid Chromatography directly on an Agilent 1100 utilizing the peak purity feature for all samples. The results are summarized below in Table 6 (% LC=% label claim).

TABLE 6

| | | Threshold | Purity |
|---|---|---|---|
| | % LC | Limit | Factor |
| Trimethoprim | | | |
| Control | 101.1 | 996.222 | 999.787 |
| Acid | 100.0 | 997.763 | 999.769 |
| Base | 100.6 | 997.145 | 999.878 |
| Sulfamethaxazole | | | |
| Control | 100.3 | 999.997 | 999.994 |
| Acid | 100.0 | 999.999 | 999.998 |
| Base | 100.0 | 999.997 | 999.994 |

The data above shows that the stress conditions do not produce loss of either trimethoprim or sulfamethoxazole for the solutions free of added excipients.

Three 4.0 mL aliquots of Formulation I is pipetted into separate 100 mL volumetric flasks. 1.0 mL of 1.0N HCl and 0.5 mL of 2.0N NaOH are added to stress. The flasks are placed at 80° C. for 3 hours, cooled and neutralized with appropriate amounts of acid or base, and brought to volume with mobile phase. The samples are filtered using a 0.45 μm nylon filter. The samples are analyzed according to the procedure immediately above.

TABLE 7

| | | Threshold | Purity |
|---|---|---|---|
| | % LC | Limit | Factor |
| Trimethoprim | | | |
| Formulation I | | | |
| Control | 88.7 | 994.794 | 999.770 |
| Acid | 91.8 | 999.969 | 999.931 |
| Base | 90.9 | 995.252 | 999.826 |
| Sulfamethaxazole | | | |
| Formulation I | | | |
| Control | 98.6 | 999.998 | 999.995 |
| Acid | 99.6 | 999.998 | 999.994 |
| Base | 102.1 | 999.998 | 999.994 |

The data above indicate that the stress conditions do not produce the loss of either trimethoprim or sulfamethoxazole in Formulation I.

The Formulations J and K of Table 5 are also tested for stability as 60° C. for three weeks. Approximately 5-20 samples of each formulation are placed in polyethylene terephthalate (PET) bottles and measured for initial purity by the same HPLC method as for the stress tests above. The samples are then heated to 60° C. for a period of three weeks and retested for impurity. The results are provided in Table 8 illustrating that the solutions are stable for at least 3 weeks at a temperature of 60° C.

TABLE 8

| | % LC | | | |
|---|---|---|---|---|
| | Initial | Initial | 3 weeks | 3 weeks |
| Sample | trimethoprim | sulfamethoxazole | trimethoprim | sulfamethoxazole |
| Formulation J | 116.5 | 106.4 | 110.0 | 103.2 |
| Formulation K | 119.2 | 107.8 | 119.6 | 106.6 |

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-74. (canceled)

75. A dosage form, comprising
a therapeutically effective amount of trimethoprim; and
a therapeutically effective amount of sulfamethoxazole;
wherein trimethoprim is present in a first dosage form and sulfamethoxazole is present in a second dosage form, or
trimethoprim and sulfamethoxazole are present in a single dosage form containing both trimethoprim and sulfamethoxazole; and
wherein the trimethoprim and sulfamethoxazole are present in a weight ratio of about 1:1 to about 1:4, trimethoprim:sulfamethoxazole.

76. (canceled)

77. A pharmaceutical kit, comprising:
the dosage form of claim 75, wherein
a plurality of the first and second dosage forms or a plurality of the single dosage forms are packaged in a container with instructions for a patient to carry out a dosing regimen for morning and evening dosing; and wherein the container optionally incorporates indicia for distinguishing between the first and the second dosage forms.

78. The kit of claim 77, wherein the dosage form is a tablet, capsule, sprinkle, candy, or liquid for oral administration.

79. The kit of claim 77, wherein the container comprises a plurality of compartments to retain the dosage forms, wherein the compartments are organized in a geometric orientation according to the dosing regimen.

80. The kit of claim 79, wherein the container is a blister pack, a blister card, a syringe, an eyedropper, or a combination comprising at least one of the foregoing containers.

81. The kit of claim 77, wherein the container comprises compartments organized for morning and evening dosing.

82. The kit of claim 77, wherein the trimethoprim is formulated in sustained-release form.

83. The kit of claim 77, wherein the first, second, or single dosage forms are packaged in dropper bottles, eyedroppers, syringes, ampules, or vials.

84. The kit of claim 83, wherein the dropper bottles, eyedroppers, syringes, ampules, or vials are pre-filled with the first, second, or single dosage forms.

85. The kit of claim 77, wherein the trimethoprim and sulfamethoxazole are present in a weight ratio of about 1:1 to about 1:3.

86. The kit of claim 77, wherein the trimethoprim and sulfamethoxazole are present in a weight ratio of about 1:1 to about 1:2.

87.-105. (canceled)

106. The dosage form of claim 75, wherein the trimethoprim and sulfamethoxazole are present in a weight ratio of about 1:1 to about 1:3, trimethoprim:sulfamethoxazole.

107. The dosage form of claim 75, wherein the trimethoprim and sulfamethoxazole are present in a weight ratio of about 1:1 to about 1:2, trimethoprim: sulfamethoxazole.

108. The dosage form of claim 75, wherein the dosage form is a tablet, capsule, sprinlde, candy, or liquid for oral administration.

109. The dosage form of claim 75, wherein the trimethoprim is formulated in sustained-release form.

110. The dosage form of claim 75, wherein the dosage form is administered to a patient to treat or prevent an upper respiratory infection, bronchitis, pneumonia, sinusitis, urinary tract infection, or otitis media; or to treat opportunistic infection in an immunosuppressed patient.

111. A method of treating or preventing an infection, comprising:
providing a dosage form to a patient, wherein the dosage form comprises
a therapeutically effective amount of trimethoprim; and
a therapeutically effective amount of sulfamethoxazole;
wherein trimethoprim is present in a first dosage form and sulfamethoxazole is present in a second dosage form, or
trimethoprim and sulfamethoxazole are present in a single dosage form containing both trimethoprim and sulfamethoxazole; and
wherein the trimethoprim and sulfamethoxazole are present in a weight ratio of about 1:1 to about 1:4, trimethoprim:sulfamethoxazole.

112. The method of claim 111, wherein the trimethoprim and sulfamethoxazole are present in a weight ratio of about 1:1 to about 1:3, trimethoprim:sulfamethoxazole.

113. The method of claim 111, wherein the trimethoprim and sulfamethoxazole are present in a weight ratio of about 1:1 to about 1:2, trimethoprim:sulfamethoxazole.

114. The method of claim 111, wherein the dosage form is administered to a patient to treat or prevent an upper respiratory infection, bronchitis, pneumonia, sinusitis, urinary tract infection, or otitis media; or to treat opportunistic infection in an imnuunosuppressed patient.

115. A dosage form, comprising
a therapeutically effective amount of trimethoprim; and
a therapeutically effective amount of sulfamethoxazole;
wherein trimethoprim is present in a first dosage form and sulfamethoxazole is present in a second dosage form, or
trimethoprim and sulfamethoxazole are present in a single dosage form containing both trimethoprim and sulfamethoxazole; and
wherein the trimethoprim and sulfamethoxazole are present in a weight ratio of about 1:1 to about 1:2, trimethoprim:sulfamethoxazole.
